(19) United States
(12) Patent Application Publication
(10) Pub. No.: US 2008/0121734 A1
(43) Pub. Date: May 29, 2008

(54) Air deodorizing system

(76) Inventor: Robert Cappellina, Caledon (CA)

Correspondence Address: Robert Cappellina, 1661 Forks of the Credit Rd, Caledon, ON, L7K 2J6, omitted

(21) Appl. No.: 11/604,426

(22) Filed: Nov. 28, 2006

(51) Int. Cl. A61L 9/04; F24F 13/02

(52) U.S. Cl. 239/44; 454/370

(57) ABSTRACT

A refillable receptacle containing scented liquid or gel is mounted on the supply side of a heating/cooling duct system of a residential, commercial or industrial building. A wick system absorbs the scented liquid or gel from the receptacle to the top of the wick. As forced air passes through the duct system, scent/fragrance from the mounted air deodorizer is pushed through the duct system and distributed to the entire building.

### FIELD OF THE INVENTION

This invention relates to the use of an air deodorizing system mounted to the duct systems of residential, commercial and industrial buildings.

### BACKGROUND OF THE INVENTION

There are many air freshening and deodorizing products in the marketplace. Manufacturers of air deodorizing products use different methods of dispensing scent/fragrance from their products, including the use of aerosol containers, heat, wick systems, battery- and electrically-powered devices. Some air deodorizing products come with more sophisticated features. They allow the users to program and control the time, duration and the desired level of scent/fragrance to be released into the atmosphere.

Examples of the different patents relating to home/building deodorizers and scent/fragrance distribution systems can be found in U.S. Pat. Nos. 6,722,529, 6,379,242, 5,924,597, 5,556,030, 5,019,352 and 4,118,226.

In U.S. Pat. No. 4,118,226, the air freshener or germicidal element is made an integral part of the filter element or support casing. U.S. Pat. No. 5,019,352 uses a combination of filter elements and wick elements in its scented air filter for use with a forced air central heating and cooling system. U.S. Pat. No. 5,556,030 uses a wicking system and heating system to cause dispersion of the fragrance throughout the desired areas. U.S. Pat. No. 5,924,597 uses solenoids and programmable timers to control the dispensing of fragrance. U.S. Pat. No. 6,379,242 is an automatic scent dispensing system that uses a duration timer to control the duration of dispensing of the scent substance and the supply of electrical power to the pump for dispensing the scent substance. U.S. Pat. No. 6,722.529 uses a pressure differential switch to sense the forced air flow in actuating a spray dispenser to discharge a freshening, deodorizing and/or disinfecting spray through a nozzle and provided hose into the ductwork, for a duration and at a repetition rate predetermined in accordance with the cleansing results desired.

The air deodorizer of the present invention uses a wick system to dispense scent/fragrance. It is designed to be easily mounted into the supply side of a heating/cooling duct system. An air deodorizing system is created when forced air from the supply side of the heating/cooling system is turned on. Unlike other air deodorizer/scent dispensers and scent dispensing systems, the air deodorizing system of the present invention provides a user-friendly, affordable and energy saving alternative to existing home/building deodorizers and scent/fragrance distribution systems.

### SUMMARY OF THE INVENTION

In the present invention is an air deodorizer comprising a receptacle; a scented liquid or gel; a wick system; a receptacle lid; a receptacle lid surface that is a magnetic panel or an adhesive material.

The wick system attached to the receptacle lid is partly immersed in the scented liquid or gel contained in the receptacle and partly exposed. When mounted on the duct system, the wick system is exposed into the duct system through an opening of the duct wall.

As forced air passes through the supply side of the heating/cooling duct system, scent/fragrance from the mounted air deodorizer is pushed through the duct system into the entire building.

### BRIEF DESCRIPTION OF THE DRAWINGS

The preferred embodiment of the present invention is described with reference to the following figures, in which:

FIG. 1 is a perspective view of the air deodorizer.

FIG. 2 is a diagram depicting the manner in which scent/fragrance from the air deodorizer mounted to the heating/cooling duct system in a building is distributed throughout the building.

### DETAILED DESCRIPTION OF THE INVENTION

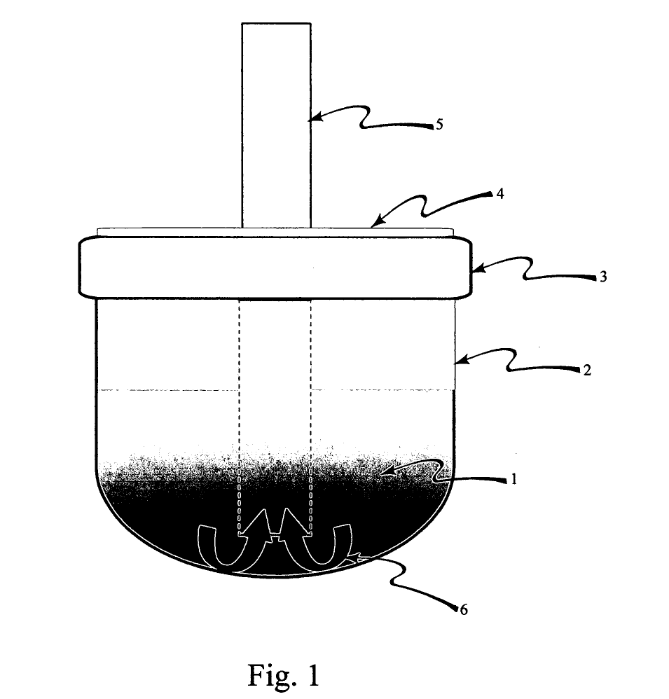

With reference to the drawings, FIG. 1 shows receptacle 2 made of plastic, glass or metal containing a scented fragrance, disinfectant, or some other odor-control substance in liquid or gel form 1. Receptacle 2 is a refillable receptacle. A receptacle lid 3 made of plastic, glass or metal prevents or reduces the evaporation of the scented fragrance 1 in the receptacle. The surface of the receptacle lid 3 is a magnetic panel or an adhesive material 4 to enable the receptacle to be mounted to the bottom face of the horizontal portion of the supply side of a heating/cooling duct system.

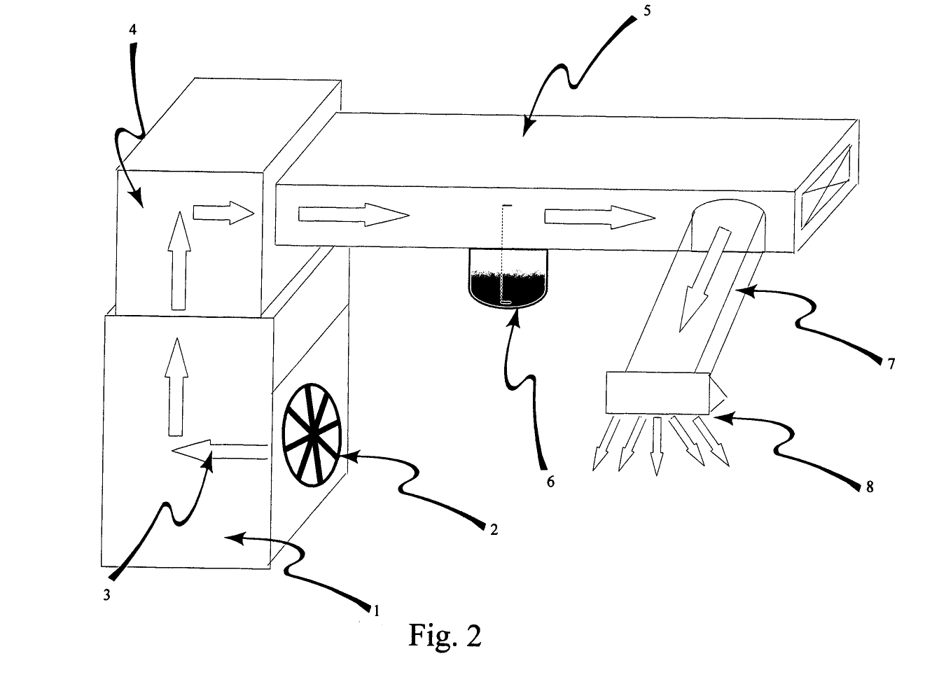

When mounted on the duct system of a building, the absorbent wick system 5 in the form of a plastic, paper, cloth, felt cloth, hollow glass or any combination thereof is used to saturate the scented fragrance, disinfectant, or other odor-control substance in liquid or gel form 1 into the wick causing the scent/fragrance to be dispersed into the duct system through an opening made on the duct wall. FIG. 2 shows how forced air 8 in the duct system distributes the scent/fragrance from the air deodorizer FIG. 1 to the entire building.

I claim:

1. An air deodorizing system comprising:
a receptacle;
a scented liquid or gel;
a wick system;
a receptacle lid with an upper surface attachable to the duct system of a building; and
a duct system.

2. The air deodorizing system of claim 1 wherein said receptacle is made of glass, metal or plastic

3. The air deodorizing system of claim 1 wherein said receptacle is refillable.

4. The air deodorizing system of claim 1 wherein said scented liquid or gel is a fragrance, disinfectant, or other odor-control substance.

5. The air deodorizing system of claim 1 wherein said wick system is a plastic, paper, cloth, felt cloth, hollow glass or any combination thereof.

6. The air deodorizing system of claim 5 wherein said wick system is partly immersed in said scented liquid or gel contained in said receptacle and partly exposed into said duct system.

7. The air deodorizing system of claim 1 wherein said main body of the receptacle lid is made of glass, metal or plastic.

8. The air deodorizing system of claim 1 wherein said surface of the receptacle lid is a magnetic panel or an adhesive material.

9. The air deodorizing system of claim 1 wherein said duct system is a heating/cooling duct system in a residential, commercial or industrial building.

10. The air deodorizing system of claim 9 wherein an opening is made to the supply side of the bottom face of the horizontal portion of said duct system.
